(19) United States
(12) Patent Application Publication
(10) Pub. No.: US 2007/0003921 A1
(43) Pub. Date: Jan. 4, 2007

(54) Method of grading disease by Fourier Transform Infrared Spectroscopy

(76) Inventor: Paul G. Andrus, Ancaster (CA)

Correspondence Address: Paul G. Andrus, 46 Wiltshire Place, Ancaster, ON, L9K 1M5, CA

(21) Appl. No.: 11/171,465

(22) Filed: Jul. 1, 2005

(51) Int. Cl. C12Q 1/00; G06F 19/00

(52) U.S. Cl. 435/4; 702/19

(57) ABSTRACT

The infrared spectrum of cells or tissues in the frequency range 900cm⁻¹-1750cm⁻¹ is digitally separated by the method of the invention into the component contributions by protein, RNA and DNA. By comparing the relative sizes of the component spectra, relative quantities of these components can be specified. The ratios protein/DNA and RNA/DNA can be used to quantify the degree of cellular biosynthesis for the purpose of grading the aggressiveness of cancer cells. The ratio nucleic acid/protein may be used to measure the nuclear cell content of blood for the purpose of quantifying the degree of systemic inflammation. The advantage of the method is in its' minimal sample size requirement and low cost.

### BACKGROUND OF THE INVENTION

With respect to malignancy, grade refers to the intrinsic aggressiveness of the cells. Tumours with higher rates of cell proliferation and/or higher tendency for invasion and metastases would be considered of higher grade. Stage refers to the degree to which a malignancy has already spread. For example, if it has breached the muscular layer of a visceral organ, or spread to regional lymph nodes or to distant organs, then it is of increasingly higher stage. While staging is objective to the extent that tumour cells can be found by radiological imaging or examination of tissue samples in the region of a tumour, grading currently depends largely upon a subjective interpretation by the pathologist. The present invention is generally aimed at measuring grade in that it examines small samples of cells or tissues. It may however assist with cancer staging in that small foci of malignancy may be picked up with automated infrared screening of tissue samples taken from lymph nodes in the region of a tumour when these foci may have been too small or overlooked by light microscopic examination. The primary aim of the invention is to provide accurate objective grading so that more informed and effective treatment decisions could be made for a given malignant tumour.

Within the infrared spectrum of cells between 900 cm⁻¹ and 1750 cm⁻¹, the Amide I band at 1650 cm⁻¹ and the Amide II band at 1544 cm⁻¹ are mostly due to protein absorbance, while the phosphodiester stretching bands at 1240 cm⁻¹ and 1084 cm⁻¹ are both mainly due to combined RNA and DNA absorbances. The dominant Amide I band region also has significant RNA and DNA absorbance as well. Fourier Transform Infrared Spectroscopy (FTIR) has been shown to differentiate lower grade from higher grade malignant lymphoma (1). The focus here was on the upwardly shifting RNA/DNA ratio with increasing grade as the contour of the symmetric phosphodiester stretching band at 1084 cm⁻¹ moves closer to the profile of pure RNA and further away from the profile of pure DNA. An increasing RNA/DNA ratio is expected with greater cellular proliferation and therefore biosynthesis. Also evident within the lymphoma spectra is an increasing level of total protein relative to total nucleic acid as suggested by higher 1650 cm⁻¹/1084 cm⁻¹ ratio and higher 1544 cr⁻¹/1084 cm⁻¹ ratio with increasing clinicopathological grade. Other studies have also shown a relative drop in nucleic acid absorbance with increasing grade of cervical and prostate cancer when these spectra were normalised to the Amide I band (2,3). This type of presentation (Amide I normalisation) is somewhat misleading in that cellular nucleic acid content is not dropping with increasing grade. In fact, cellular RNA content and therefore total nucleic acid content is rising with grade. It has been shown by flow cytometry that for cells in culture, regardless of whether or not they are cycling (proliferating), the ratio of protein/RNA is remarkably constant (4). This is referred to as “balanced growth”. Therefore with balanced growth it would be expected that protein content rises with RNA relative to DNA, and so protein rises relative to RNA+DNA. This explains the relative decrease in total nucleic acid compared to total protein with increasing grade as protein rises at a faster rate than RNA+DNA. The present invention disclosure is the first to connect the changes within the 1084 cm⁻¹ nucleic acid band (i.e. at 1121 cm⁻¹) due to increasing RNA/DNA ratio, with the changes in protein/nucleic acid ratio, and links them together along the cellular differentiation continuum.

Another advantage of “connecting” the protein related spectral changes to the RNA/DNA changes, beyond verifying internal biological consistency (balanced growth/biosynthesis where RNA/DNA and protein/DNA rise proportionately (4)), occurs when FTIR microscopy is used to examine small clusters of cells from paraffin imbedded tissue sections. In this case the signal to noise ratio for the RNA related changes at 1121 cm⁻¹ may be poor rendering RNA/DNA ratio analysis less reliable (see for example prostate cancer (3)). However the dominant combined nucleic acid peak at 1084 cm⁻¹ is relatively strong even for small cell clusters. Consequently protein/nucleic acid ratio changes at 1650 cm⁻¹/1084 cm⁻¹ or 1544 cm⁻¹/1084 cm⁻¹ can be used as biosynthetic parameters for cancer grading even when the overall spectral signal is relatively weak. Even though in the prostate cancer spectra there are greater apparent differences elsewhere (1150 cm⁻¹-1480 cm⁻¹), the signal at 1084 cm⁻¹ most precisely separates the two higher Gleason grades from the two lower, and would be most reliable for this purpose. This higher “noise” away from the 1084 cm⁻¹ peak becomes evident by examining the cervical cancer spectra (2) where measured stepwise change with grade does not occur in the region 1150 cm⁻¹-1480 cm⁻¹, but clear changes at 1084 cm⁻¹ are maintained and are similar in magnitude to those for lymphoma (1) and prostate cancer (3). The cellular sample was large enough in the cervical cancer spectra (2) for the 1121 cm⁻¹ RNA pulse to be seen just like that for lymphoma (1). Similar consistent changes are apparent in spectra of H-ras transfected fibroblasts (5). The present method is also superior to grading methods that rely upon increasing consumption of energy substrates such as glycogen, in that the biomolecules measured (protein, RNA, DNA) have a universally applicable trend across various tissue/cell origins that works no matter what the malignant grade. Glycogen however is consumed early along the de-differentiation continuum, and many tissues/cells are glycogen-poor to begin with.

Recent discovery of micro-RNA interference of messenger RNA as a possible fundamental mechanism in the regulation of normal cellular differentiation and in the development of malignancy (6,7), supports focusing on the quantification of the protein products of biosynthesis as a universally applicable method to gauge malignant grade. Treatments aimed at restoring micro-RNA interference of m-RNA, and thereby “downgrading” a malignancy to a benignancy could be readily monitored by the method of the invention. In this model of cancer in which the biosynthetic machinery is relatively unregulated, even the diagnosis of cancer might be thought of as graded cut points of biosynthetic velocity. Some entities previously called cancer may potentially be reclassified as functionally benign.

Others have proposed using the broad based relative decrease in the nucleic acid region of the spectrum as a cancer grading parameter for cervical (2), prostate(3) and colon cancer (Mordechai U.S. Patent Application No. 20050017179, Jan. 27, 2005). The present disclosure is however the first to explain the biochemical basis for this universal trend, and therefore to propose a more accurate and reliable cancer grading test that directly measures the biomolecular analytes which are responsible for it. The present method recognises that there are numerous parameters that could be chosen to quantify grade based on the teaching of the invention that protein and RNA absorbance rise relative to DNA absorbance with increasing biosynthesis and cancer grade. The invention specifies however that any chosen parameter should be based on knowledge of the individual component absorbances, as opposed to using broader spectral regions which are less specific and therefore less accurate in that they are more likely to include variable biomolecular mixtures. This more informed and direct approach of the present invention ensures that the “test” measures the same thing every time regardless of the sample's tissue of origin, and allows for verification that significant quantities of undesired biomolecules (for example glycogen or glycoproteins) are not included in the measurement. Both of these aspects are important for a test of cancer grading to be sufficiently trustworthy for widespread clinical use.

The absorbance ratio 1540 cm⁻¹/1080 cm⁻¹, or conversely 1080 cm⁻¹/1540 cm⁻¹ has been previously proposed as a diagnostic marker to distinguish chronic leukemia form normal lymphocytes (8). The Amide II band at 1540 cm⁻¹ is mostly due to protein absorbance. This historical work does not disclose or imply the method of the present invention. It recognised that chronic leukemic cells contained greater amounts of nucleic acid relative to protein when compared to normal lymphocytes, and concluded that relatively more nucleic acid was attributable to the malignant transformation and a proliferative impulse. The present invention disclosure teaches that the opposite trend is actually true in that the proliferative impulse is indicated by a decrease in nucleic acid absorbance relative to protein absorbance. Chronic leukemia is unusual in that it is an indolent type of cancer that lingers for many years, and is not susceptible to treatment precisely because of its lack of biosynthetic activity, given that chemotherapies target cycling cells.

Dukor RK (U.S. Pat. No. 6,841,388) describes a spectral marker derived from the spectrum of tumour extracellular material. The present invention generally focuses on the spectrum of the intracellular material, however it is recognised that within tumour tissue, extracellular material occurs between cells and will necessarily be included within the scanned area. This is not considered a problem in that the present invention focuses on the biosynthetic machinery of the tissue and its' products, so that on which side of the cell membrane these products lie within a cluster of tumour cells is not considered critical within the scope of the present invention.

With respect to inflammation, there are many clinical situations where precise and rapid quantification of the severity or degree of acute or chronic systemic inflammation will aid treatment or triage decisions. Acute life threatening bacterial illness can be difficult to separate from less serious illness in busy emergency rooms. Patients vary widely in their perception and representation of symptoms. An objective, rapid and inexpensive measure of acute systemic inflammation would improve triage accuracy. Chronic inflammation is now recognised as the basis for many chronic diseases including vascular disease. Measures of chronic inflammation such as white blood cell counts and C-reactive protein levels have been correlated with risk for future cardiovascular disease events such as heart attack or stroke (9). In this version of the present invention, the nucleic acid content of peripheral blood is compared to the protein content of peripheral blood, which would include haemoglobin. As the white blood cell count rises, the nucleic acids found within the white blood cells rise in total absorbance relative to haemoglobin and other blood proteins. There is no requirement to separate the white cells from the blood. The advantage of the method of the invention over other methods of measuring inflammation is in its' minimal sample, time, and cost.

### BRIEF DESCRIPTION OF THE DRAWINGS

FIG. 1 is a depiction of infrared spectra in the frequency range 900 cm⁻¹-1750 cm⁻¹ showing the component spectra and the sample spectrum. Relative spectral shifts with changing cellular differentiation are shown.

FIG. 2 shows infrared spectra in the frequency range 900 cm⁻¹-1750 cm⁻¹ for malignant lymphoma tumours of varying clinicopathological grades.

FIG. 3 shows infrared spectra in the frequency range 900 cm⁻¹-1150 cm⁻¹ for malignant lymphoma tumours of varying clinicopathological grades.

FIG. 4 shows infrared spectra in the frequency range 1100 cm⁻¹-1150 cm⁻¹ for malignant lymphoma tumours of varying clinicopathological grades.

FIG. 5 is a depiction of an infrared spectrum in the frequency range 900 cm⁻¹-1160 cm⁻¹ for pure RNA, pure DNA and an example sample spectrum having an RNA/DNA ratio of 4:1.

FIG. 6 illustrates various alternative ways of assigning an index value to the overall spectral absorbance for an individual component spectrum.

FIG. 7 is a depiction of an infrared spectrum in the frequency range 900 cm⁻¹-1160 cm⁻¹ for pure RNA, pure DNA, low grade (indolent) lymphoma, high grade (aggressive) lymphoma, showing 1121 cm⁻¹ absorbance values (taken from actual spectra (1)(10)) expressed as a fraction of 1084 cm⁻¹ absorbance (X), and the method of obtaining the RNA/DNA ratio for the lymphomas.

FIG. 8 is a depiction of an infrared spectrum in the frequency range 900 cm⁻¹-1750 cm⁻¹ showing the approximate relative changes in the component and combined spectra between low and high grade lymphoma.

### SUMMARY OF THE INVENTION

The infrared spectrum of an unknown minimal sample of cells or tissue from a malignant tumour is digitally separated into relative component spectral contributions by protein, RNA and DNA. The component spectra are then quantified with respect to their relative absorbances. The cellular content ratios RNA/DNA and protein/DNA are used as a measure of cellular biosynthesis. More aggressive and proliferative cancers (higher grade) have higher protein synthesis. This biosynthetic trend also correlates inversely with cellular differentiation. Cells approaching senescence have decreasing protein synthesis. Another version of the invention uses the ratio (RNA+DNA)/protein of peripheral blood to quantify the white blood cell content as a measure of systemic inflammation from a minimal sample such as a drop of blood as yielded by a lancet commonly used by diabetics to test blood sugar.

### DETAILED DESCRIPTION OF THE METHOD OF THE INVENTION

An infrared spectrum in the frequency range 900 cm⁻¹-1750 cm⁻¹ is obtained from the unknown sample of cells. The methods of FTIR microspectroscopy are well developed and described elsewhere.

A reference spectrum for pure RNA and pure DNA (10) are obtained and stored in digital memory.

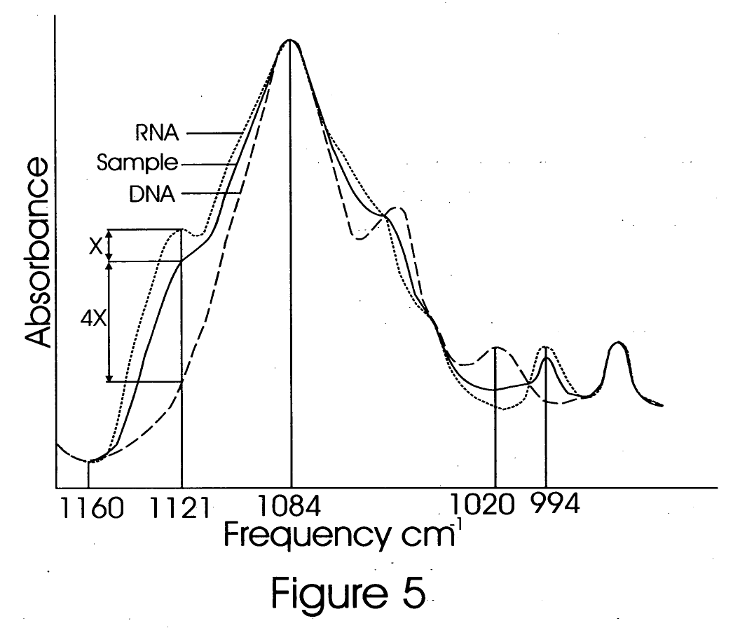

The symmetric phosphodiester stretching band centered at 1084 cm⁻¹ is the result of absorbance by RNA and DNA with minimal contribution by protein. Spectra in the frequency range 900 cm⁻¹-1160 cm⁻¹ for the unknown sample, reference RNA, and reference DNA are compared at one or more wavenumbers where RNA and DNA most differ such as 1121 cm⁻¹. By normalising all three spectra by max-min at 1084 cm⁻¹ and 1160 cm⁻¹, the sample spectral absorbance at 1121 cm⁻¹ will lie below that of RNA and above that of DNA. The relative distance of the sample absorbance from that of each of the pure nucleic acids at 1121 cm⁻¹ varies inversely with their relative contributions to the sample spectrum. For example if the sample spectrum is one quarter the distance from RNA as from DNA at 1121 cm⁻¹, then the sample has four parts RNA to one part DNA (FIG. 5). A combined RNA+DNA spectrum can be generated from the reference spectra by adding the four times the RNA spectrum to one times the DNA spectrum. The combined spectrum should nearly exactly match the sample spectrum in the region 900 cm⁻¹-1160 cm⁻¹. “Impurities” if present would be apparent at this point as the combined nucleic acid spectrum would not match that of the sample. The most common other biomolecule that absorbs in this region is glycogen at 1030 cm⁻¹ and 1050 cm⁻¹, however it is usually substantially all consumed and therefore not present in malignant spectra. However, a reference spectrum for glycogen could be used to subtract glycogen absorbance from the sample spectrum in the region to leave a sample spectrum that lies on the continuum between that of pure DNA and that of pure RNA. Software that allows adding and subtracting of spectra from one another in various proportions is standard in FTIR spectroscopy.

Now that a combined RNA+DNA spectrum has been generated which matches the sample spectrum between 900 cm⁻¹-1160 cm⁻¹, the combined nucleic acid spectrum is then normalised to the sample spectrum at 1084 cm⁻¹, and then subtracted from the sample spectrum leaving a normalised protein spectrum as the result. This generated protein spectrum could be compared against known protein spectra to ensure no unexpected “impurities”.

At this point all three components of the sample: protein, RNA and DNA, have individual spectra “normalised” in size relative to one another. In the example given above, the RNA spectrum is four times the absorbance of the DNA spectrum at 1084 cm⁻¹, and the protein spectrum size is fixed by assuming no significant protein absorbance at 1084 cm⁻¹ within the sample spectrum.

The ratio protein/DNA directly measures the product of biosynthesis and therefore is the preferred parameter for this purpose. However it is recognised within the scope of the invention that other ratio parameters will rise in keeping with the method of the invention. Example grading parameters where relative quantities of protein (P), RNA (R), and DNA (D) are compared include the following: R/D, R/(R+D), P/D, P/(R+D), (P+R)/D, (P+R)/(R+D), (P+R+D)/D, (P+R+D)/(R+D).

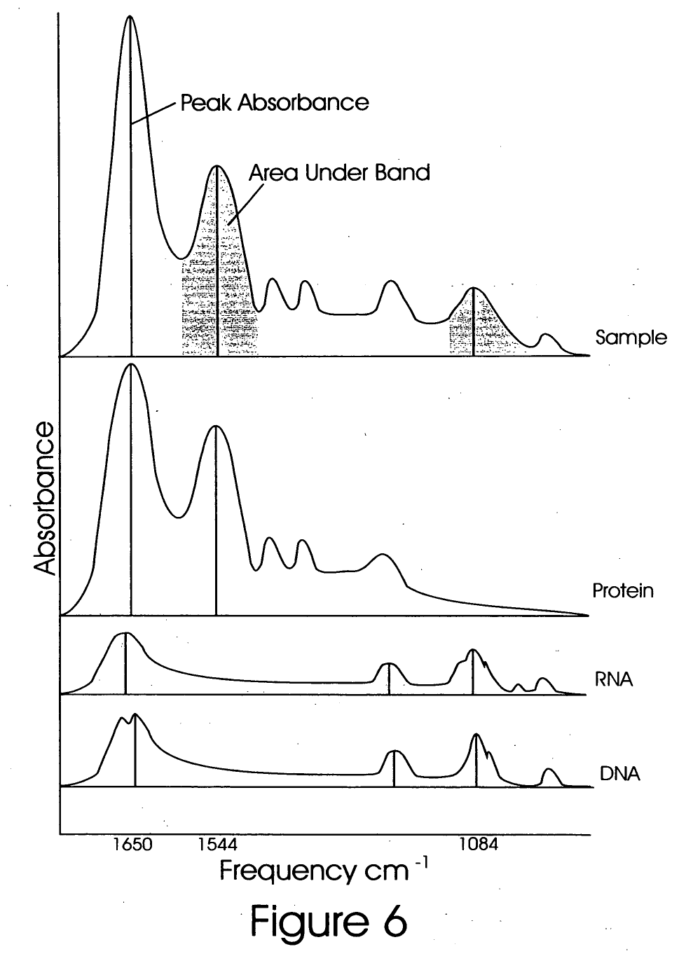

There are many ways to assign a value to the quantity of each component spectrum. One could use the single absorbance at one or more of the dominant peaks, or the area under one or more of the bands, or the area under the entire spectrum in the region studied 900 cm⁻¹-1750 cm⁻¹(FIG. 6). Ultimately since the component spectra all have a fixed spectral pattern, that is the peaks don't shift relative to each other in size (this is not entirely true as different proteins may have differing Amide I and Amide II contributions), it doesn't matter much which bands or wavenumber regions are chosen for quantification as long as the same ones are used to compare results between samples. Using the larger peaks would however be more sensible in that a higher signal to noise ratio would be present here.

It is recognised within the scope of the invention that parameters could be pre-selected based on the teaching of the invention with respect to the biomolecular content trends, without completely separating each sample spectrum into component spectra over the entire range 900 cm⁻¹-1750 cm⁻¹ for every test. For example P/(R+D) could be measured directly by peak absorbance at 1544 cm⁻¹ divided by peak absorbance at 1084 cm⁻¹, or (P+R+D)/(R+D) could be measured by peak at 1650 cm⁻¹ divided by peak at 1084 cm⁻¹. This approach amounts to selective spectral separation, and is an obvious variation of the method of the invention. These readily obtained ratios vary with biosynthesis, but are weaker correlates of actual biosynthesis when RNA/DNA and thus protein/DNA are high as in higher grade malignancy. It is recognised in the present disclosure that by understanding the specific biomolecular analytes that one wants to measure, using band peak maximum absorbance at 1084 cm⁻¹ as a quantifying measure of that analyte (total nucleic acid) is superior to using the area under a band or the area under a broader spectral region for the following reasons. The band peak is the most direct and strongest signal of that analyte. The absorbances on the shoulders of a band are completely dependant on the peak, and therefore do not add spectral information to the peak absorbance. On the contrary these regions away from the peak but within the band area may contain undesired minor “contaminants” (i.e. those due to carbohydrates at 1030 cm⁻¹, 1050 cm⁻¹, 1155 cm⁻¹ and 1170 cm⁻¹) that lessen the accuracy of the desired true measurement.

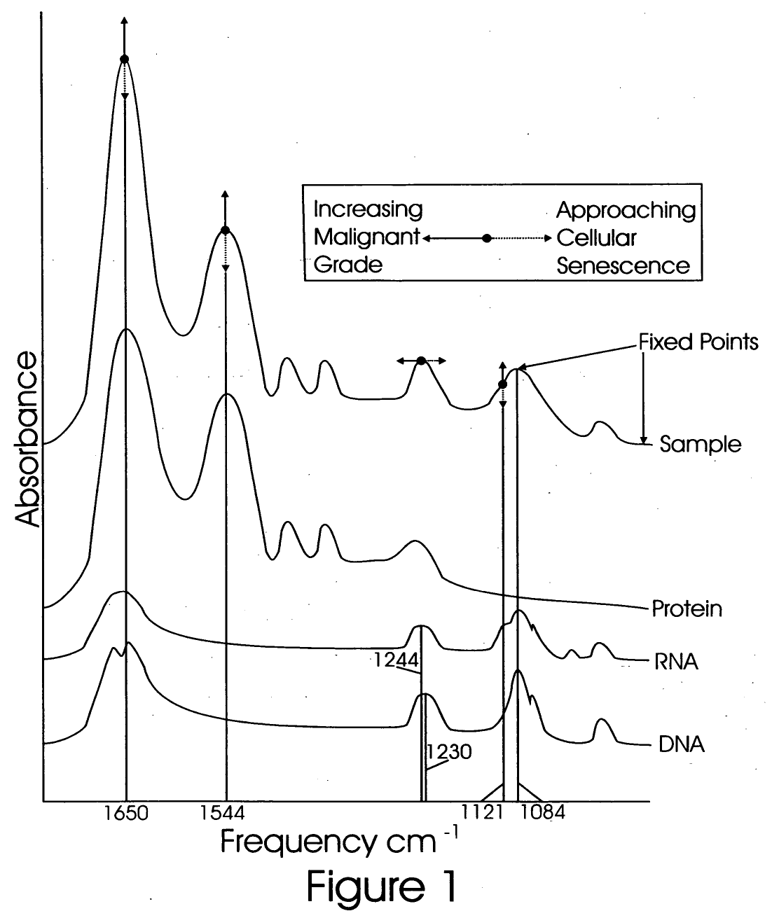

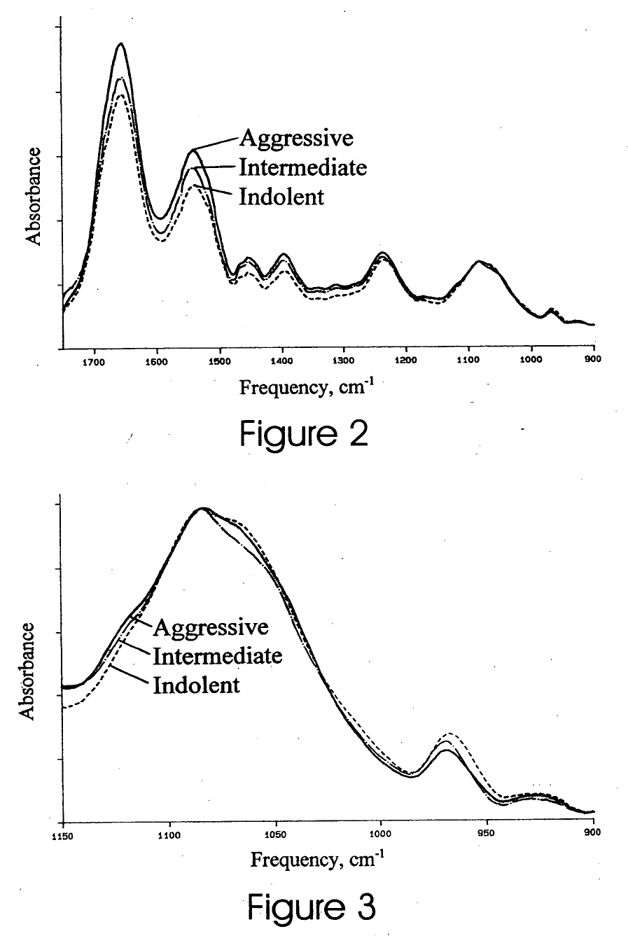

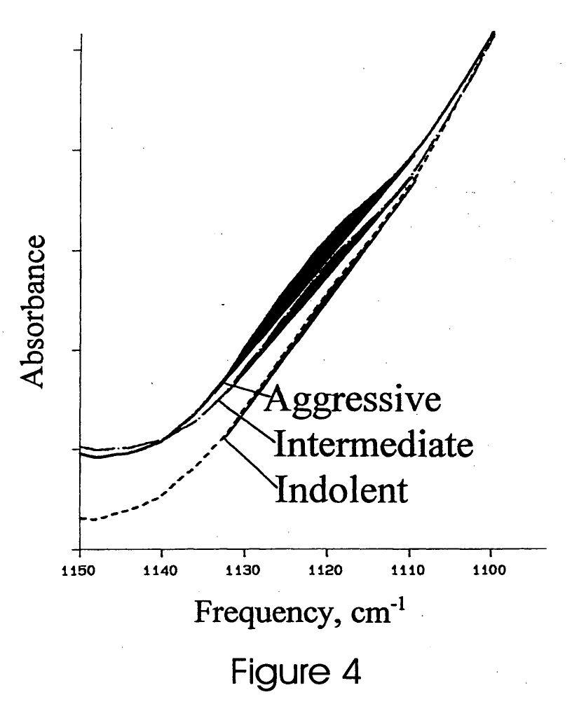

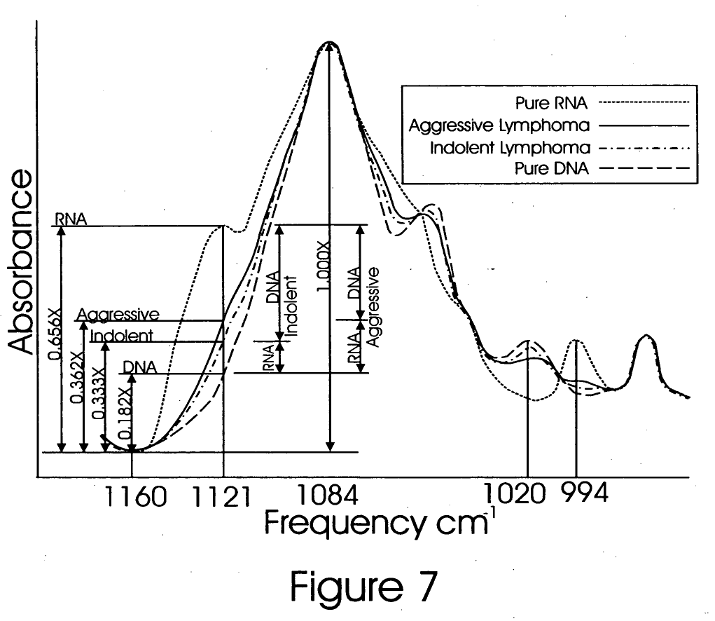

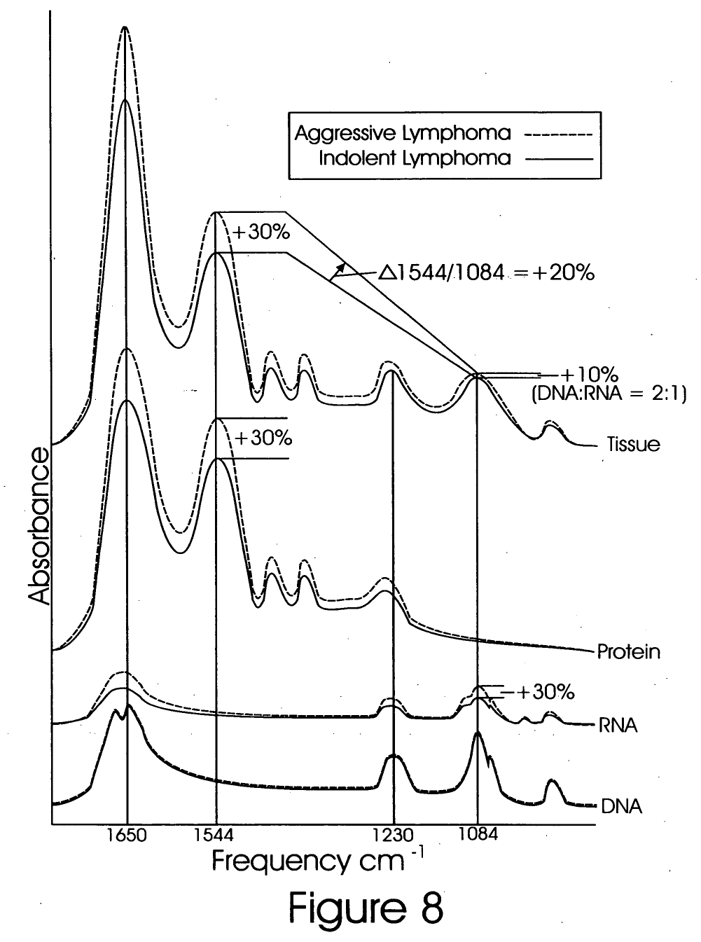

As a check of the internal consistency of the method, examination of specific malignant lymphoma (FIGS. 2,3,4) spectra reveals the following. In order to determine the RNA/DNA ratio, the region 1084 cm⁻¹-1160 cm⁻¹ can be used as it spans a local maxima to a local minima in the spectrum of RNA and DNA with maximum RNA-DNA difference in the middle at 1121 cm⁻¹ owing to the RNA specific absorbance at 1121 cm⁻¹ (FIG. 7). The ratio of absorbance differences (A1121-A1160)/ (A1084-A1160) quantifies what fraction of the way between the minima at about 1160 cm⁻¹ and the maxima at 1084 cm⁻¹ is the absorbance at 1121 cm⁻¹. For pure RNA from reference spectra (10), the ratio (A1121-A1160)/(A1084-A1160) is 0.656. For pure DNA it is 0.182. Therefore for the low grade lymphoma with the ratio (A1121-A1160)/(A1084-A1160) of 0.333, an RNA/DNA ratio of 0.469 can be calculated, and the high grade lymphoma with the ratio (A1121-A1160)/(A1084-A1160) of 0.362, an RNA/DNA ratio of 0.612 can be obtained. The RNA content rises by the ratio 0.612/0.469 or 30.5% in going from low to high grade. RNA+DNA rises by the ratio 1.612/1.469 or 10%. Therefore the protein band at 1544 cm⁻¹ should rise relative to the RNA+DNA band at 1084 cm⁻¹ by 30.5%-10%=20.5% (FIG. 8) (assuming balanced growth where RNA and protein rise by the same percentage (4)). In fact the 1544 cm⁻¹ band rises by about 21% relative to the 1084 cm⁻¹ band going from low grade to high grade (measured from actual lymphoma spectra in FIG. 2) in keeping with internal consistency within the spectrum and suggesting balanced biosynthesis where RNA and protein rise proportionately relative to DNA with increasing lymphoma grade. This analysis of course uses DNA content as the basis, whether or not DNA content changes on a cellular basis as it would with aneuploidy. The cell boundaries are ignored as the method examines only relative changes in RNA and protein compared to DNA along the cellular differentiation continuum (FIG. 1). This cellular differentiation model of the present disclosure may shed light on changes within the spectra of white blood cells during acute infection (11) in which the white cells appear to be less biosynthetically active than normals. Possibly the infectious stimulus pushes the white cells to end differentiate and approach senescence in performing their final bacterial fighting function.

With respect to changes at 1121 cm⁻¹, the acute infection white blood cells show relatively little change compared to normals because the RNA/DNA ratio of normal white cells is already very low at about 0.3, so that their 1084 cm⁻¹-1160 cm⁻¹ profile already looks much like that of pure DNA. In further differentiating, the 1084 cm⁻¹-1160 cm⁻¹ profile does however move even closer to that of pure DNA as seen by the concavity which develops in the 1084 cm⁻¹-1160 cm⁻¹ region of the acute infection white cells (11), similar to the concavity of this region seen in the spectral profile of DNA (10). Similarly, when RNA/DNA is already high, further de-differentiation (and increased RNA/DNA) yields relatively smaller changes in the appearance of the 1084 cm⁻¹-1160 cm⁻¹ region as the spectrum approaches that of pure RNA more slowly, the nearer it gets to pure RNA. An example of this occurs with H-ras trasfected fibroblasts (5). It follows that the greatest rate of change at 1121 cm⁻¹ with change in differentiation, occurs when RNA/DNA is 1. If only a small cluster of cells are scanned, the signal to noise ratio may be too weak at 1121 cm⁻¹ to be used reliably as discussed above.

The present disclosure describes two strategies for measuring systemic inflammation which do not obviously follow from the prior acute infection work (11). The present method recognises the need to avoid having to separate blood components (white cells, red cells, plasma) in order for the test to be sufficiently rapid and inexpensive (better than a simple blood count). To this end, the present method uses the spectrum of whole blood (white cells, red cells and plasma combined) to estimate the white blood cell count. The blood proteins, found mostly in red cells (haemoglobin) and plasma (albumin . . . ) are used as the basis here, making use of the fact that white cells rise in number relative to red cells with inflammation in general. The second strategy of the present method for measuring inflammation uses the analysis of cellular biosynthesis/differentiation as described above. This is essentially the same as the method of cancer grading except that it moves in the opposite direction with respect to differentiation. One significant difference however with the cancer analysis is that because whole blood is used, comparison between protein content and nucleic acid content cannot be used to indicate differentiation because most of the protein comes from outside (red cells, plasma) of the nuclear cells (white cells). In other words the nucleic acids and proteins are not connected biosynthetically within whole blood as they are within separate cell populations (cancer cells, white cells separated from whole blood . . . ). Consequently, the differentiation analysis for whole blood uses only the intrinsic nucleic acid changes at 1121 cm⁻¹ as described above.

I claim:

1. A method of using Fourier Transform Infrared Spectroscopy for measuring the malignant grade of a sample of cells comprising the steps of:
Obtaining an infrared absorbance spectrum of said cell sample; separating said sample spectrum into a protein component spectrum, and a total nucleic acid component spectrum; quantifying each of said component spectral absorbances yielding index values of the relative content of protein, and total nucleic acid within said sample of cells;
wherein the index value of protein rises relative to the index value of nucleic acid with increasing malignant grade of said cells.

2. A method as in claim 1 wherein the quantification of component spectral absorbances is achieved by choosing one or more band peak maximum absorbances, or the area under one or more absorbance bands within each of the component spectra, as representative of the overall absorbance of each component.

3. A method as in claim 1, wherein differences in the absorbance patterns of RNA and DNA within the frequency range 900 cm⁻¹-1150 cm⁻¹ are used to determine the relative absorbance contributions of RNA and DNA to said total nucleic acid absorbance spectrum, thereby allowing determination of the RNA/DNA content ratio and index values of DNA and RNA within said sample of cells; wherein said RNA/DNA ratio rises with increasing malignant grade of said cells.

4. A method as in claim 3, wherein differences in the absorbance values of RNA and DNA at 1121 cm⁻¹ are used to determine the relative absorbance contributions of RNA and DNA to said total nucleic acid absorbance spectrum, thereby allowing determination of the RNA/DNA content ratio and index values of DNA and RNA within said sample of cells; wherein said RNA/DNA ratio rises with increasing malignant grade of said cells.

5. A method as in claim 3, wherein any of the following ratios between said index values of protein (P), RNA (R), and DNA(D) are chosen: RID, R/(R+D), P/D, P/(R+D), (P+R)/D, (P+R)/(R+D), (P+R+D)/D, (P+R+D)/(R+D); wherein said chosen ratio rises with increasing malignant grade of said cells.

6. A method as in claim 1, wherein the absorbance band centered at 1544 cm⁻¹ within the sample spectrum is taken as the protein component spectrum, and the absorbance band at 1084 cm⁻¹ is taken as the total nucleic acid component spectrum.

7. A method as in claim 1, wherein the absorbance band centered at 1650 cm⁻¹ within the sample spectrum is taken as the protein component spectrum, and the absorbance band at 1084 cm⁻¹ is taken as the total nucleic acid component spectrum.

8. A method as in claim 1, wherein a reference spectrum is obtained for a biomolecule which is not from the group: protein, RNA, DNA; said biomolecule having significant absorbance within the frequency range of said infrared absorbance spectrum of said sample, whereby said reference spectrum is digitally subtracted from said infrared absorbance spectrum of said sample.

9. A method as in claim 8 wherein said biomolecule is glycogen.

10. A method of using Fourier Transform Infrared Spectroscopy for measuring the degree of systemic inflammation from a sample of whole blood cells comprising the steps of:
Obtaining an infrared absorbance spectrum of said cell sample; separating said sample spectrum into a protein component spectrum, and a total nucleic acid component spectrum; quantifying each of said component spectral absorbances yielding index values of the relative content of protein, and total nucleic acid within said sample of cells;
wherein the index value of nucleic acid rises relative to the index value of protein with increasing white blood cell count or the degree of systemic inflammation

11. A method as in claim 10 wherein the quantification of component spectral absorbances is achieved by choosing one or more band peak absorbances, or the area under one or more absorbance bands within each of the component spectra, as representative of the overall absorbance of each component.

12. A method as in claim 10, wherein the absorbance band at 1544 cm⁻¹ within the sample spectrum is taken as the protein component spectrum, and the absorbance band at 1084 cm⁻¹ is taken as the total nucleic acid component spectrum.

13. A method as in claim 10, wherein the absorbance band at 1650 cm⁻¹ within the sample spectrum is taken as the protein component spectrum, and the absorbance band at 1084 cm⁻¹ is taken as the total nucleic acid component spectrum.

14. A method of using Fourier Transform Infrared Spectroscopy for measuring the degree of systemic inflammation from a sample of whole blood cells comprising the steps of:
Obtaining an infrared absorbance spectrum of said cell sample; determining the relative absorbance contributions of DNA and RNA to said spectrum by using differences in the absorbance patterns of DNA and RNA within the frequency range 900 cm⁻¹-1150 cm⁻¹, thereby allowing determination of the DNA/RNA content ratio within said sample of cells; wherein said DNA/RNA ratio rises with greater differentiation of said peripheral blood cells during conditions of greater systemic inflammation.

15. A method as in claim 14, wherein differences in the absorbance values of DNA and RNA at 1121 cm⁻¹ are used to determine the relative absorbance contributions of DNA and RNA to said spectrum, thereby allowing determination of the DNA/RNA content ratio within said sample of cells; wherein said DNA/RNA ratio rises with greater differentiation of said peripheral blood cells during conditions of greater systemic inflammation.
